(19) United States
(12) Patent Application Publication
(10) Pub. No.: US 2025/0160961 A1
(43) Pub. Date: May 22, 2025

(54) ALIGNMENT AND DISTORTION COMPENSATION OF REFERENCE PLANES USED BY SURGICAL DEVICES

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Charles J. Scheib, Loveland, OH (US); Kevin M. Fiebig, Cincinnati, OH (US); Luis Andrade Fernandez, JR., La Honda, CA (US)

(21) Appl. No.: 18/810,208

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/20; A61B 6/00; A61B 8/08; A61B 90/00; A61M 25/01

(52) U.S. Cl. CPC A61B 34/20; A61B 6/5258; A61B 8/5269; A61B 2034/2051; A61B 2034/2065; A61B 2090/364; A61M 2025/0166

(57) ABSTRACT

Devices, systems, and techniques for alignment and distortion compensation of reference planes used by surgical devices. A first surgical instrument may include a processor and an electromagnetic (EM) sensor. The first surgical instrument may sense, using the EM sensor, a predicted location of the EM sensor. The predicted location may be distorted by an EM field. The first surgical instrument may receive, from a second surgical instrument, a coordinate system associated with imaging of an area around the EM sensor. The first surgical instrument may determine an adjusted location of the EM sensor based on the received coordinate system.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/810,230, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

In operating rooms, multiple surgical imaging devices may operate in close proximity to one another. An imaging device may have a sensor that tracks the location of the imaging device. Other devices in the operating room may create electromagnetic fields that affect the accuracy of the sensor's ability to track the imaging device. The devices may not be aware of the presence of other devices. Even if the devices are aware of other devices, the devices may not be able to communicate information such as information related to electromagnetic distortion. Without this knowledge, a user (e.g., surgeon) may not know the actual location of an imaging device, which may affect the user's ability to safely perform the operation.

To enable such devices to detect and compensate for distortion, a common reference plane may be created for multiple imaging streams. A reference plane from a first imaging system may be used as a means to compensate for distortion (e.g., electromagnetic distortion) of coordinates by a second imaging system. Multiple oblique reference planes may be aligned and distortion compensation may be performed for at least one of the sensed locations. A first coordinate system may be derived from real-time measurements from a sensor. The distortion compensation may use a second coordinate system originating from an independent system to form the basis for the common coordinate system for both local reference planes.

The first coordinate system may be used to determine the current location of a flexible endoscope distal end. The first coordinate system may accumulate additive errors due to distortion of the measurement. The first and second coordinate systems may be aligned by associating the first system with the second system and compensating for the distortion caused by the second system's measurements (e.g., relative to the first imagine system's detector). The two systems may be aligned using local re-calibration of the flexible endoscope. The distortion correction may involve measuring the first sensor location and the current field distortion measured by at least one other separate sensor (e.g., a redundant sensor). The redundant sensor may be located at a distance from the first sensor. The distance between the sensors may be greater than the size of the patient.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 illustrates an example situationally aware surgical system.

FIG. 5 illustrates an example surgical operating room with robotic arms.

FIG. 6 illustrates an example of electromagnetic distortion caused by metal staples.

FIG. 7 illustrates example operations and communication by two surgical devices.

FIG. 8 is a block diagram illustrating example components of a surgical device.

FIGS. 9A-D illustrate graphical representations of magnetic distortion and distortion compensation.

FIG. 10 illustrates an example of distortion compensation using sensors in an operating room.

FIGS. 11A and 11B illustrate example distortion cause by a CT machine moving around a patient and example distortion compensation.

FIG. 12 illustrates an overlayed comparison of a sensed location and an adjusted location of an endoscope.

FIG. 13 illustrates examples of a low bandwidth reference plane and a high bandwidth reference plane.

FIG. 14 illustrates an example method that may be performed by a surgical instrument.

### DETAILED DESCRIPTION

A more detailed understanding may be had from the following description, given by way of example in conjunction with the accompanying drawings.

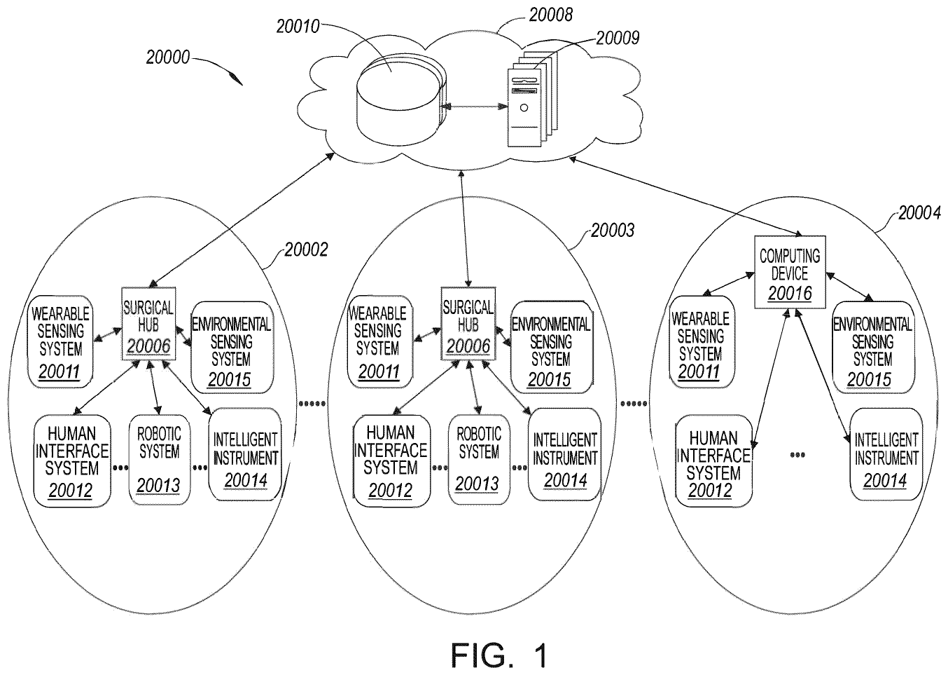

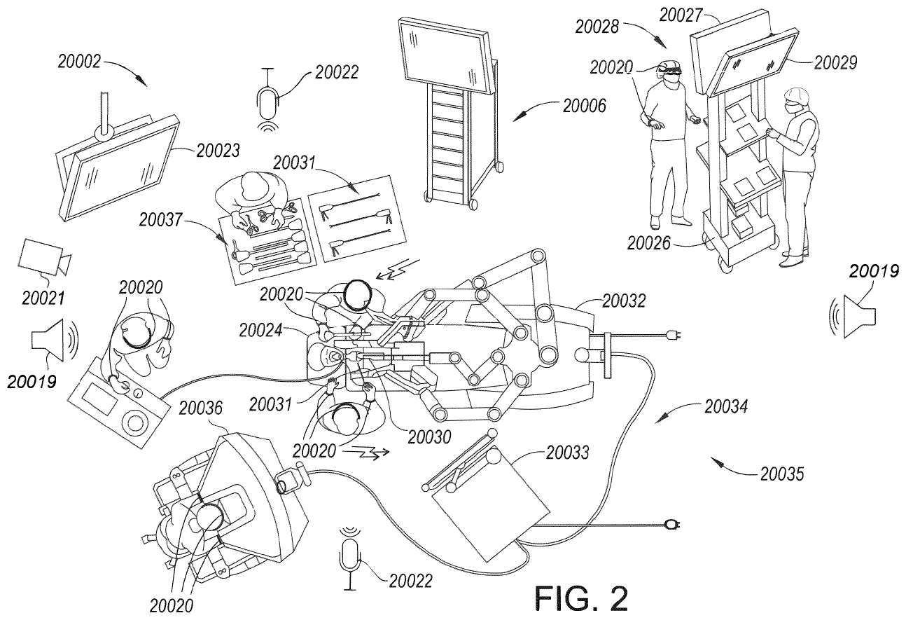

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

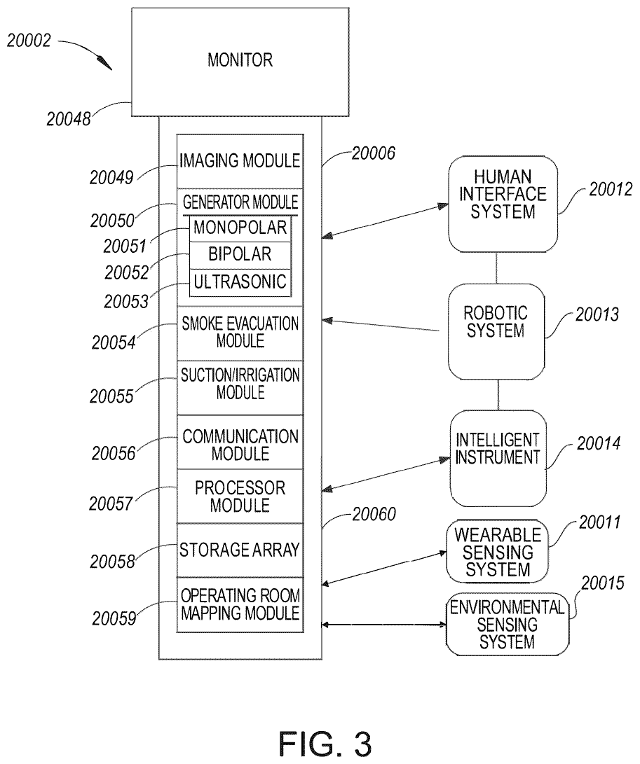

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

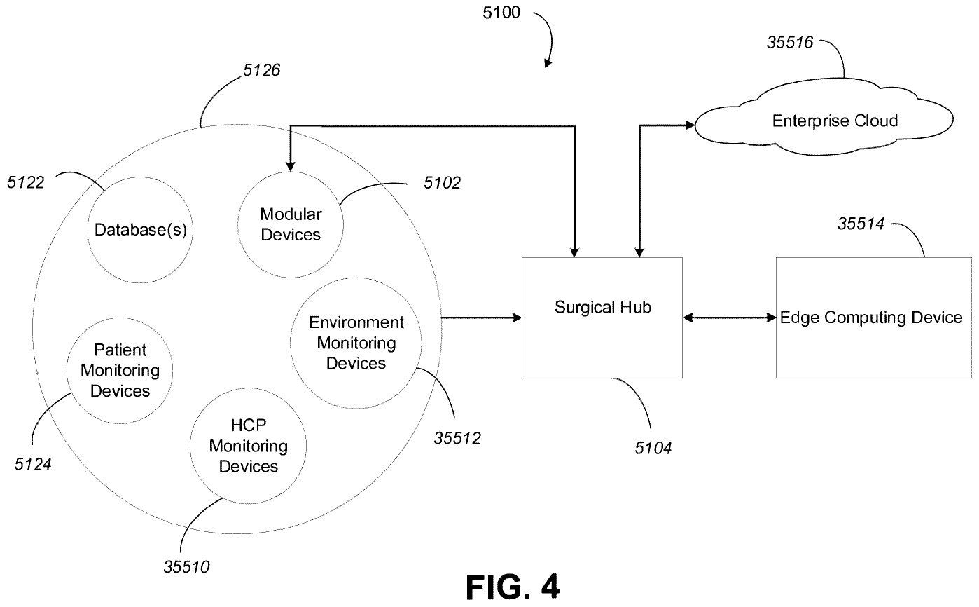

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

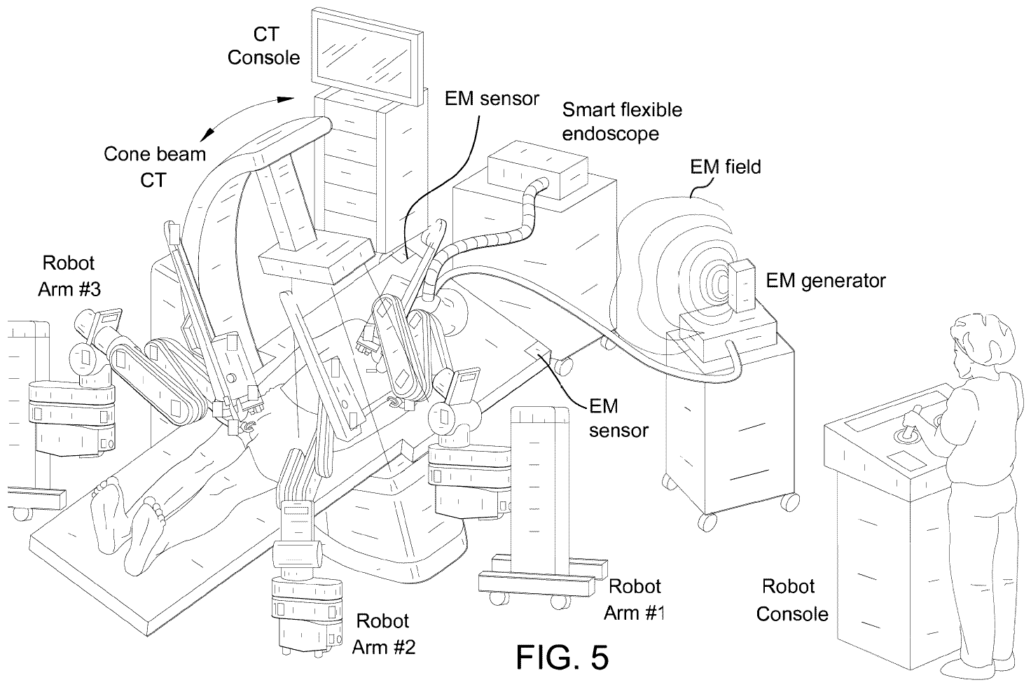

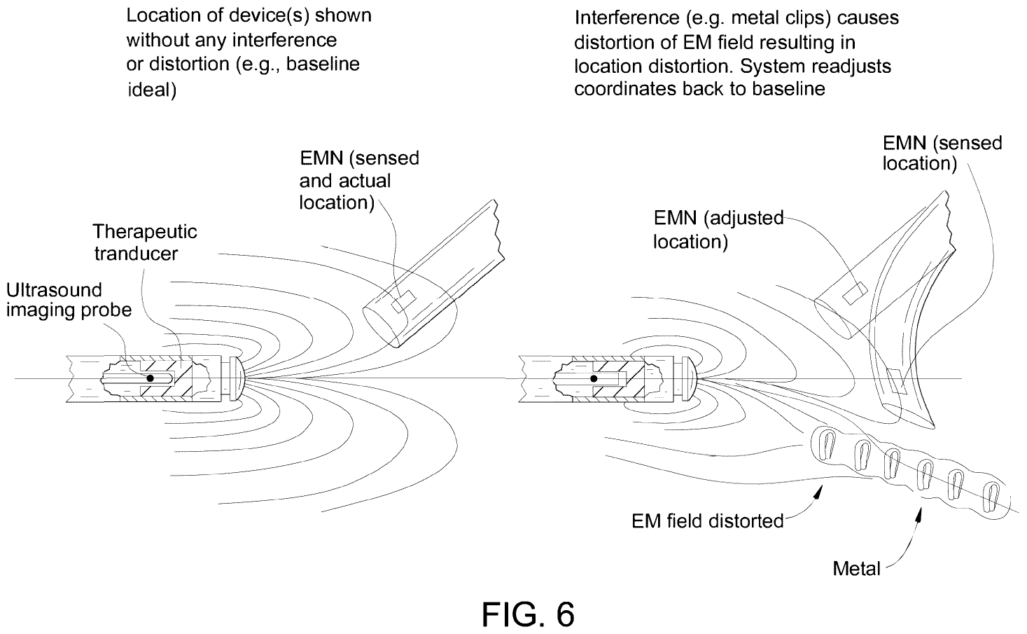

In operating rooms, multiple surgical imaging devices may operate in close proximity to one another. FIG. 5 illustrates an example surgical operating room with several different devices. An imaging device may have a sensor that tracks the location of the imaging device. Other devices in the operating room may create electromagnetic fields that affect the accuracy of the sensor's ability to track the imaging device. FIG. 6 illustrates an example of electromagnetic distortion caused by metal staples/clips. As shown, the metal may distort the EM field. The distortion may impact the EM navigation (EMN) device. For example, the metal may cause the EMN sensed location to be skewed from the actual location of the EMN device.

For example, if a first surgical instrument with an EM sensor is near an ultrasound imaging probe and a metal object (e.g., a row of metal staples or clips), the EM sensor may experience distortion of a predicted location of the EM sensor. The EM field causing distortion of the predicted location may be created by proximity of the ultrasound imaging probe to the metal object. The first surgical device may determine an adjusted location of the EM sensor based on the received coordinate system by aligning expected anatomy associated with the predicted location with anatomy in the area around the EM sensor associated with the received coordinate system.

The devices may not be aware of the presence of other devices. Even if the devices are aware of other devices, the devices may not be able to communicate information such as information related to electromagnetic distortion. Without this knowledge, a user (e.g., surgeon) may not know the actual location of an imaging device, which may affect the user's ability to safely perform the operation.

To enable such devices to detect and compensate for distortion, a common reference plane may be created for multiple (e.g., two) imaging streams. For example, the common reference plane may be created using a reference plane from one imaging system as a means to compensate for a distortion of coordinates of another imaging system. Alignment of two oblique reference planes and distortion compensation for at least one of the sensed locations may be realized by utilizing another coordinate system originating from an independent system. For example, the other coordinate system may be used to form the basis for a common coordinate system for both reference planes). FIG. 6 further illustrates an example of the adjusted location of the EMN device (e.g., after distortion compensation).

The first coordinate system may be derived from the real-time measurement of a sensor (e.g., in three dimensional space). The first coordinate system may be used to determine the current sensed/measured location of the sensor. The first coordinate system may accumulate additive errors (e.g., due to distortion of the measurement).

An example device may sense, using the EM sensor, a predicted location of the EM sensor. The predicted location may be distorted by an EM field. The device may receive, from a second surgical instrument, a coordinate system associated with imaging of an area around the EM sensor. The device may determine an adjusted location of the EM sensor based on the received coordinate system.

The alignment of the two coordinate systems may rely on an association between the first system and the second system. The alignment of the two coordinate systems may rely on the distortion compensation of the second system's measurements (e.g., relative to the first imagine system's detector).

The reference planes may have a relative commonality. One plane may have a higher validity (e.g., due to a more closely controlled association to the patient-related reference plane(s). A multi-level reference plane may be used to enable local and global registration. A sub or local reference plane may be formed within a linked global reference plane. A multi-level reference plane may be generated (e.g., to provide maximum available data to the user).

Multi-level data transfer may be established. The data transfer may enable registration of smart systems (e.g., including “simple” and/or “complex” registration). The simple registration may include minimal data points. The simple registration may include critical structures and enough data to render a general visual rendering. The complex registration may include the simple registration as a base. The complex registration may overlay the simple registration with the data available in that smart system (e.g., to include specific and detailed anatomy and/or blood flow). If the system shares the registration, the simple registration data (e.g., at least the simple registration data) may be transferred. Different levels of registration data may also be provided (e.g., on top of the simple registration data, for example, based on the capability of the receiving smart system).

An example surgical instrument may receive registration information of registered elements on a second surgical instrument. The surgical instrument may monitor positional changes of the second surgical instrument based on the registration information. The surgical instrument may adjust the adjusted location of the EM sensor based on the monitored positional changes.

For example, the surgical instrument may be a laparoscopic probe with an EM sensor. The EM sensor may be a magnetic tracker. The second surgical instrument may be a moveable CT machine. The EM field causing distortion of the predicted location may be created by proximity of the moveable CT machine to the first surgical instrument. The surgical device may determine the adjusted location of the EM sensor based on the received coordinate system by aligning expected anatomy associated with the predicted location with anatomy in the area around the EM sensor associated with the received coordinate system.

Feature(s) associated with creating a common global reference plane are provided herein. Multiple (e.g., two) systems may dynamically exchange their reference planes with each other (e.g., including the location of each other). A global reference plane may be established within each system. A device may determine an adjusted location of the EM sensor with respect to the global reference plane (e.g., that is associated with an operating room in which the first surgical instrument is located).

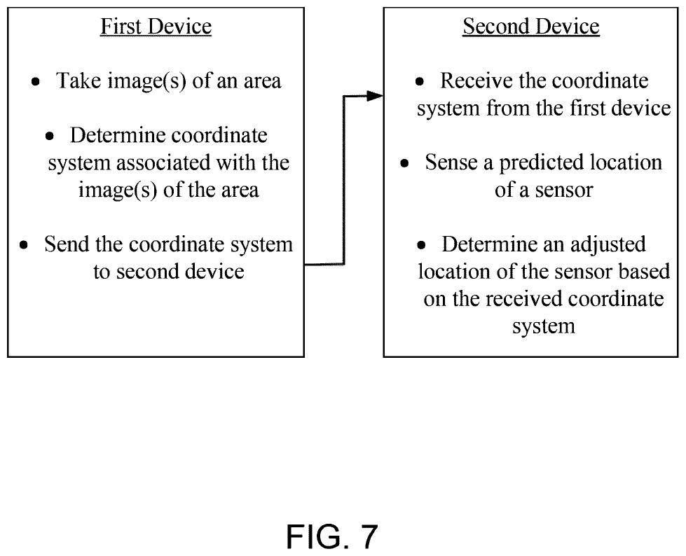

FIG. 7 illustrates example operations and communication by two surgical devices. As shown, a first device may capture image(s) of an area. The first device may determine a coordinate system associated with the image(s) of the area. The first device may send the coordinate system to a second device. The second device may receive the coordinate system. The second device may sense a predicted location of a sensor. The second device may determine an adjusted location of the sensor based on the received coordinate system.

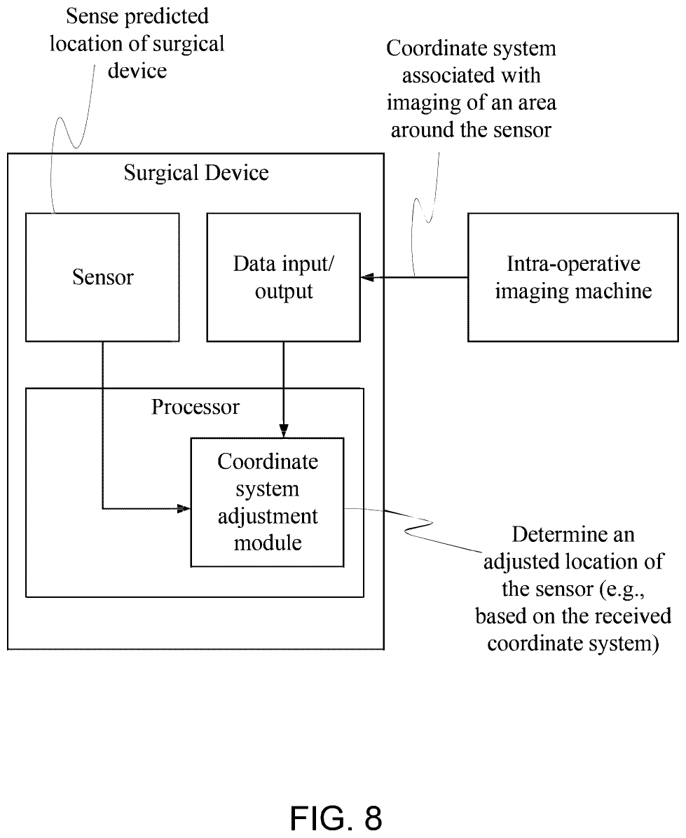

FIG. 8 is a block diagram illustrating example components of a surgical device. As illustrated, an intra-operative imaging machine may capture image(s) of a surgical area (e.g., around a sensor on another device). The intra-operative imaging device may send, to a surgical device, a coordinate system associated with the image(s) of the surgical area. For example, the coordinate system may indicate a reference geometry used by the intra-operative imaging device.

The surgical device may receive the coordinate system (e.g., via a data input/output module). The surgical device may include a sensor (e.g., the sensor around which the intra-operative imaging device is capturing images). The surgical device may use the sensor to sense a predicted location of the surgical device. However, as described herein, the sensed location may be distorted from the actual position due to EM interference. The surgical device may therefore input the sensed location and the coordinate system into a coordinate system adjustment module. The coordinate system adjustment module may determine an adjusted location of the sensor based on the coordinate system from the intra-operative imaging device.

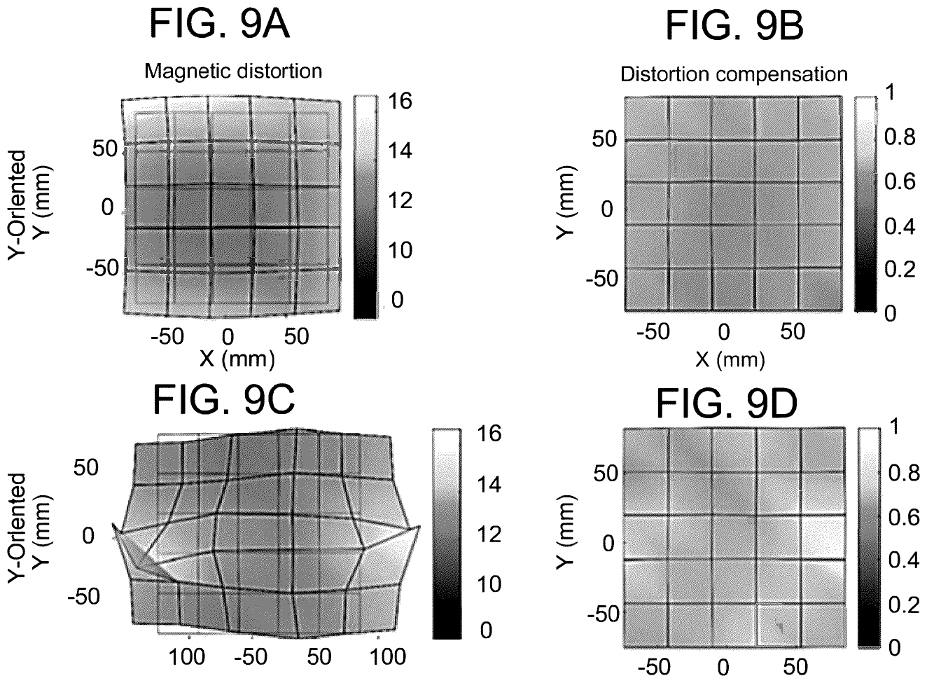

FIGS. 9A-D illustrate graphical representations of magnetic distortion and distortion compensation. FIGS. 9A and 9C illustrate the distortion of a square electromagnetic coordinate system. The distortion may be caused by the presence, movement, or introduction of metal objects in close proximity to the transmitter or sensor (e.g., within inches). If the system measures the distortion from another point of view or secondary sensing system, the system may compensate for the magnetic field distortion (e.g., with algorithms, for example, rather than merely changing calibration of the sensing system). FIGS. 9B and 9D illustrate examples of distortion compensation for the magnetic distortion shown in FIGS. 9A and 9C, respectively. The distortion compensation may be represented by a transform function. This transform function may be adapted with additional sensed input(s) (e.g., to more accurately compensate for the distortion).

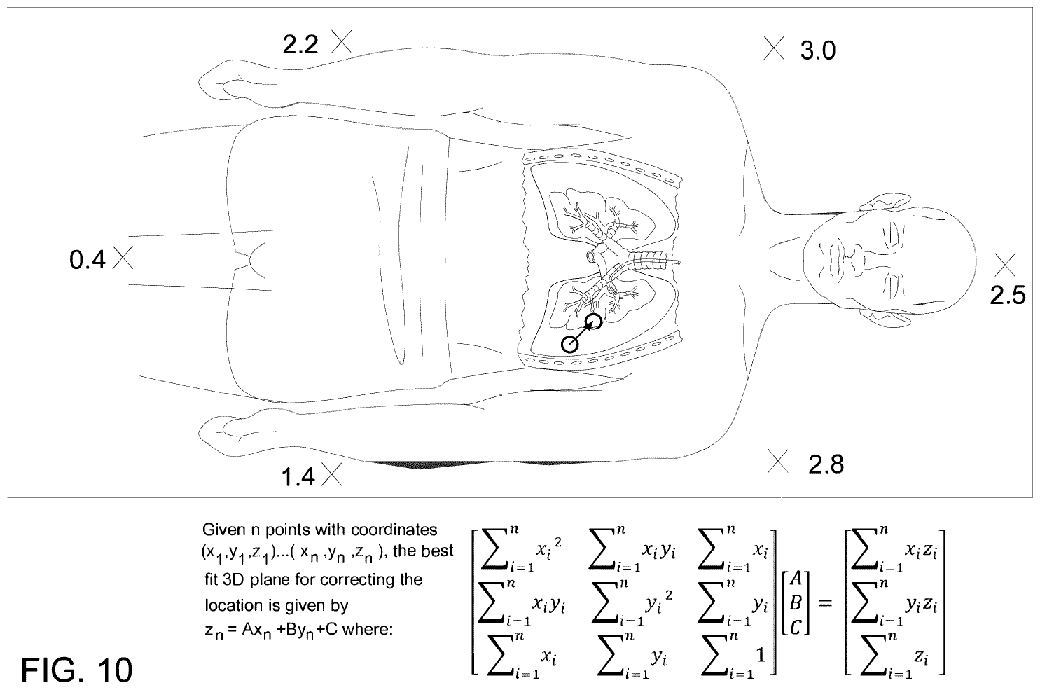

A (e.g., single) physical lattice may be placed on and/or around the patient (e.g., as illustrated in FIG. 10). A (e.g., each) system may utilize a global reference plane from the physical lattice. Feature(s) associated with determining the orientation of the lattice are provided herein. An absolute position reference sensor may be used to determine the Z-orientation of the lattice. The lattice may include one or more (e.g., multiple) sensors (e.g., wireless beacons). The sensors may include a relatively predefined geometry. A sensor may be given a certain level of precedence/priority. Each sensor may include a unique identity. The unique identity may provide a known relative location. The other items referencing the relative location may be able to establish their own orientation (e.g., relative to that fixed point). The absolute orientation of the lattice may become irrelevant (e.g., once the other items establish their own orientation).

A (e.g., single) system may be used as the global coordinate system. The other system(s) may be established based on the global coordinate system. A sensor may be placed on a first system. A tracker (e.g., minion) may track the sensor (e.g., thereby allowing the tracker to adapt its system to the first system to form a unified reference plane).

For example, FIG. 10 illustrates an example of distortion compensation using sensors in an operating room. As shown, the operating table may have multiple (e.g., six in this example) sensors. The sensors may surround the patient. For example, as shown, there may be a sensor by the patient's head, several sensors on either side of the patient's torso, and/or a sensor near the base of the patient's torso. Although not shown, additional sensors (e.g., near the patient's feet) may be used as well.

Each sensor may detect a certain magnitude and direction of EM distortion. For example, the sensor at the top of the patient's head may detect a distortion magnitude of 2.5 (e.g., which may be a percentage of distortion relative to a base-level of distortion). The sensor near the patient's right shoulder may detect a distortion magnitude of 3.0 (e.g., slightly higher than that at the first sensor). The sensors may be used to determine a 3D distortion plane. For example, given n points with coordinates (x₁, y₁, z₁) . . . (xₙ, yₙ, zₙ), the best fit 3D plan

$$[ \begin{matrix}{\sum }_{i = 1}^{n} {x}_{i}^{2} & {\sum }_{i = 1}^{n} {x}_{i} {y}_{i} & {\sum }_{i = 1}^{n} {x}_{i} \\ {\sum }_{i = 1}^{n} {x}_{i} {y}_{i} & {\sum }_{i = 1}^{n} {y}_{i}^{2} & {\sum }_{i = 1}^{n} {y}_{i} \\ {\sum }_{i = 1}^{n} {x}_{i} & {\sum }_{i = 1}^{n} {y}_{i} & {\sum }_{i = 1}^{n} 1\end{matrix} ] [ \begin{matrix}A \\ B \\ C\end{matrix} ] = [ \begin{matrix}{\sum }_{i = 1}^{n} {x}_{i} {z}_{i} \\ {\sum }_{i = 1}^{n} {y}_{i} {z}_{i} \\ {\sum }_{i = 1}^{n} {z}_{i}\end{matrix} ] 3 {y}_{n} + C$$

where

Magnetic distortion may be caused by proximity of adjacent metallic objects within a typical OR environment. The magnetic distortion may result in 3 millimeters to 45 millimeters positional error in the EMN system of the robotic flexible endoscope. Endoscopic navigation location and orientation (e.g., of an endoscopic EMN system) may be sensed. For example, the endoscopic navigation location and orientation may be sensed using ultrasound. The endoscopic navigation location and orientation may be sensed using a force-strain measurement of the flexible scope drive.

Errors (e.g., distortion) may be sensitive to position and orientation of metal objects within the EM field and the relative location of the sensors to the impact-generating objects. The field distortion may change non-linearly as the metal objects move relative to the sensors and the emitter.

Predefined traceable calibration markers with room instrumentation may be used to monitor the 3D space of the room, tools, and movements within the room. A 3D assay of the OR may be monitored (e.g., by utilizing predictable calibrations and registration strategies).

A first coordinate system may be used to relate the current location of the flexible endoscope distal end (e.g., as it moves along a path, for example, a path defined by a pre-operative mapping of the hollow passages). For example, the distortion introduced by a fluoroscopy C-arm CT machine within the operating field near the table and patient may be a source of magnetic field distortion. A hybrid navigation framework (e.g., where EMN is used for continuous navigation and X-ray is delegated to on-demand use during delicate maneuvers or for confirmation) may be used. In this case, the X-ray machine may be a source of metallic distortion for the EMN system. The nature of the distortion may depend on the C-arm position (e.g., cannot be predetermined).

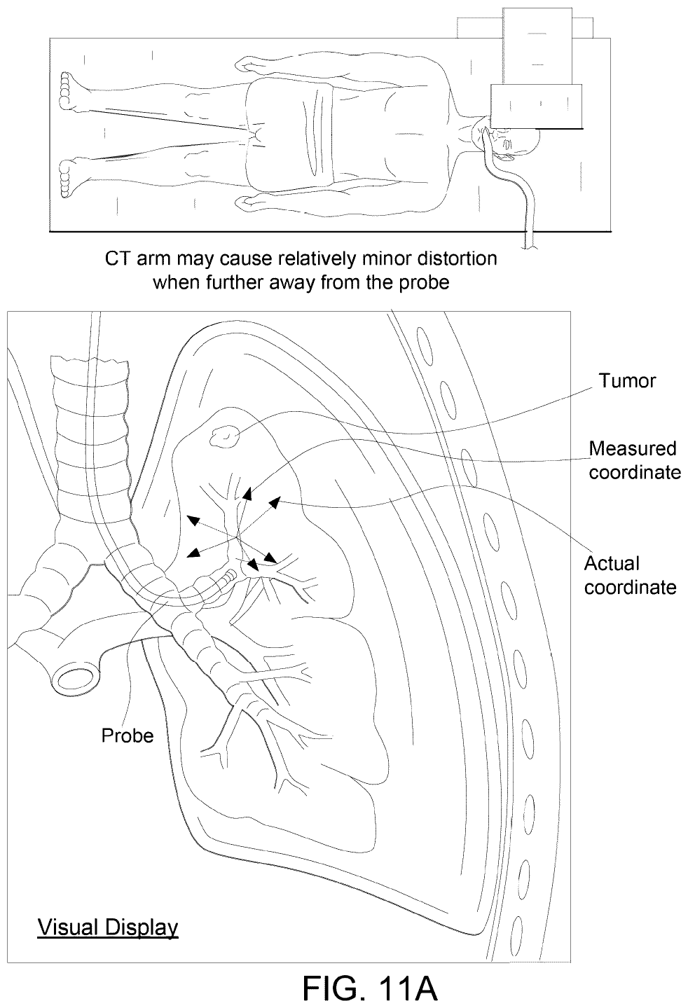

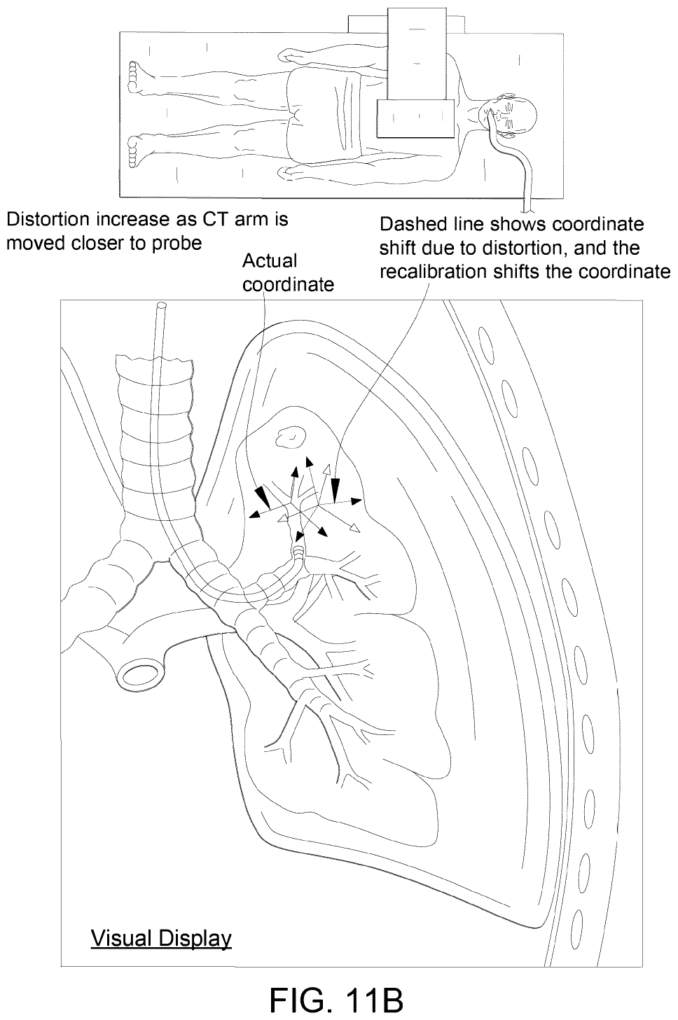

In an example, a robotic flexible scope may determine a position of its tip (e.g., based on the insertion of the scope and additive errors based on time and surrounding metal objects). A cone-beam CT arm movement amplify the error as it moves into place to recalibrate. FIGS. 11A and 11B illustrate example distortion cause by a CT machine moving around a patient and example distortion compensation. The distorted and compensated coordinate locations of a sensor on an endoscopic probe are illustrated with coordinate systems. The scope may monitor distortion caused by proximity of the second surgical instrument (e.g., CT machine). The scope may identify a change in position of the CT machine. The distortion caused by proximity of the CT machine may be reduced due to the change in position. The scope may adjust the adjusted location of the EM sensor based on the reduced distortion.

As illustrated in FIG. 11A, while the CT machine is near the patient's head, the sensor may be impacted by relatively minor distortion. As shown, the flexible endoscopy local coordinate system may deviate from the global anatomy of the body. For example, as shown, the distorted coordinate system may be skewed counterclockwise from the original coordinate system (e.g., without distortion), but the origin of the coordinate systems may be close to the same. The cone-bean CT may be used to determine where the local coordinate system should be. The cone-beam CT may communicate the true alignment of the local coordinate system to the flexible scope (e.g., for the scope to correct its system). The realignment of the coordinates may realign the expected anatomy to that of the real anatomy.

A illustrated in FIG. 11B, as the CT arm is moved into position over the patient's chest, the distortion experienced by the sensor may increase (e.g., because the source of the distortion is closer). As illustrated by the dashed coordinate system, the measured location of the sensor may be skewed counterclockwise from the original coordinate system and shifted to the right. This may be problematic if the surgeon believes that the endoscope is in another bronchial tube. For example, the surgeon may incorrectly determine a location of a tumor and make an unnecessary incision.

The coordinate axis with white arrows in FIG. 11B illustrates a first part of a transform to compensate for the distortion. As shown, the coordinate axis with white arrows is a clockwise-shifted version of the measured coordinates. As a second part of the transform, the coordinate axis with white arrows may be shifted to the left until it is aligned with the actual coordinate axis.

If the CT is moved away, the movement may impact the signal (e.g., again). This may cause misalignment. The monitored misalignment as the cone-beam CT approached (e.g., the misalignment between FIGS. 11A and 11B) may be used as a template to computationally adjust for the return CT motion. Because the local flexible scope coordinates were monitored as the CT arm was brought into position, the system may use this as an estimate of the CT arm interference with the electromagnetic field. The estimate of the CT arm interference may be used to auto-compensate for the same adjustment as the CT arm is removed from the field (e.g., leaving the flexible scope with a true position of its coordinate system).

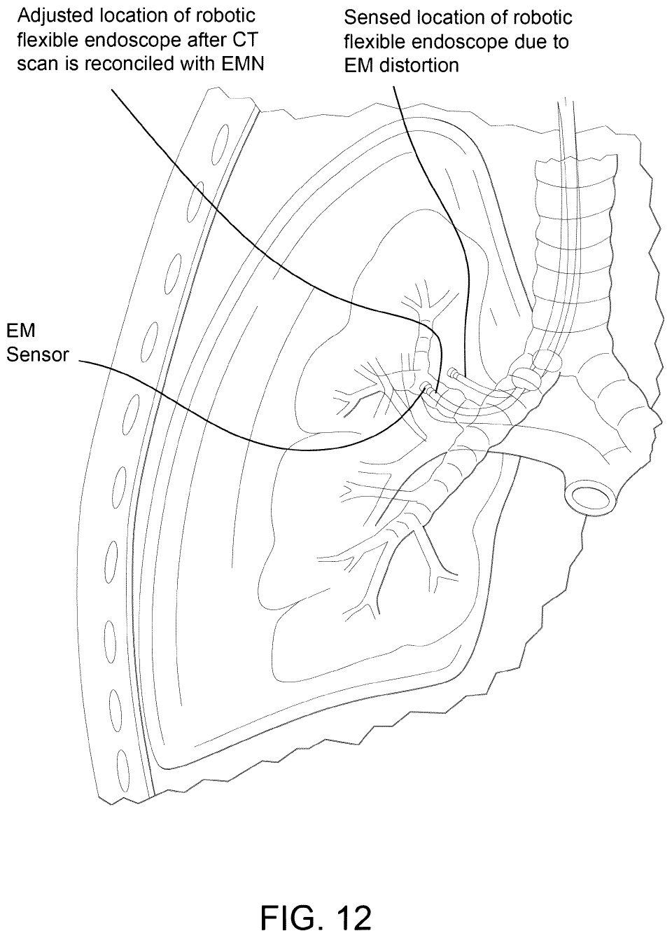

In an example, a pre-operative CT scan of the patient may be a reference plane (e.g., scanned in the supine position). The current (e.g., intraoperative) patient position (e.g., lateral position) may be the actual current global reference plane. The CT (e.g., cone beam CT) may be positioned by the surgeon. The CT may establish a local reference plane. The EMN of the flexible endoscope may create another local reference plane. A device (e.g., a Hugo robot) may have a (e.g., single) integrated reference plane. The integrated reference plane may be made of a plurality of (e.g., five) local reference planes (e.g., one for each arm of the device). FIG. 12 illustrates an overlayed comparison of a sensed location and an adjusted location of an endoscope.

An example device may receive EM field monitoring information from a monitoring device located at a first distance from an EM device causing the predicted location of the device to be distorted (e.g., by an EM field). The distance may be greater than a second distance between the device and the EM device. The device may adjust the predicted location of the EM sensor based on the EM field monitoring information. The distortion compensation/alignment may involve a local recalibration of the flexible endoscope electromagnetic navigation (EMN). The correction of the distortion of the EMN field sensing may be based on measurements at the sensor location and the current field distortion measured by at least one other separate (e.g., redundant) sensor. The second redundant sensor may be located at a distance from the first sensor. The distance may be greater than the size of the patient.

The C-arm for the cone beam CT may have registration elements on it. The registration elements may enable the cameras in the room to detect location, movement, and relationships to the EMN (e.g., and enable a better compensation factor for the EMN). The cameras may be enhanced with Lidar (e.g., to make accurate distance measurements in a 3D space with the spatial relationships handled by the imaging of the room visually). Predefined calibration stickers or markers may be used to reflect IR light or give the device a perspective measure (e.g., by the inclusion of several inter-related squares on each marker to allow the cameras to determine their angles with respect to the markers).

Externally-applied magnets (e.g., closer to the patient and in multiple 3D locations) may minimize the re-calibration interference caused by the C-arm. A more complete 3D assay of the room, measurement devices in the room, and the devices' relational location may be used to provide an improved in-situ calibration of the EMN (e.g., on-the-fly calibration).

Multiple imaging systems and coordinate systems may be used. A common reference plane for two imaging streams may be created (e.g., by utilizing a reference plane from a first imaging system as a means to compensate for a distortion of coordinates by a second imaging system). For example, a first imaging system may produce a laparoscopic view towards the uterus during a hysterectomy. A second imaging system (e.g., external to the patient) may track a fiducial system (e.g., a three-ball fiducial system) on the end of a uterine manipulator. The external image system may be registered to a global reference frame (e.g., by another three-ball system). The registration may be provided to the robot with a laparoscopic view. Kinetic chain errors may be reduced by using a three-ball fiducial system on laparoscopic or robotic instruments.

Reference planes from two separate visualization sources (e.g., including orientation) may be harmonized. Magnetic field navigation/tracking recalibration or distortion correction (e.g., on the fly magnetic field navigation/tracking recalibration or distortion correction) may be used. A smart system may adapt AR visualization to provide recalibration during data distortions.

Stereotactic surgery is a minimally invasive form of surgical intervention that makes use of a three-dimensional coordinate system to locate small targets inside the body. The system may then perform a procedure (e.g., ablation, biopsy, lesion, injection, stimulation, implantation, radiosurgery, etc.) on the targets. Predictable calibration and registration techniques may be used in stereotactic image-guided interventions. The predictable calibration and registration techniques may be used to minimize the burden of actively sensing distortion (e.g., distortion created by the interaction between the metal of the calibration system and the EMN).

In an example, an ultrasound (3D ultrasound) system may be used to achieve augmented reality (AR) visualization during laparoscopic surgery (e.g., for the liver). To acquire 3D ultrasound data of the liver, the tip of a laparoscopic ultrasound probe may be tracked inside the abdominal cavity (e.g., using a magnetic tracker). The accuracy of magnetic trackers may be affected by magnetic field distortion that results from the close proximity of metal objects and electronic equipment (e.g., which may be unavoidable in the operating room).

A temporal calibration may be used to estimate a time delay (e.g., between a first system moving and a second system receiving coordinate information for the first system). The temporal calibration may be integrated into the motion control program of the motorized scope control (e.g., to enable artifact magnitude identification that may be used to limit the magnitude's effect on the physical measurement of position).

Redundant electromagnetic field monitoring may be used. The EMF monitoring may be performed by a second magnetic sensor that is positioned at a distance from the primary source. The redundant measurements may be effected differently than the primary source by the metallic in the vicinity. The field distortions may be identified by the comparison of the two measurements. The identified field distortions may be minimized (e.g., removed) from the primary measurement.

Feature(s) associated with an endoscopic to laparoscopic clocking orientation multi light system are provided herein. The clocking of a flexible endoscope working channel may be aligned. The system may determine the orientation of the working channel and the camera of the flexible endoscopy device (e.g., relative to the laparoscopic view). This may provide proper coordination of the two systems. In an example, an endoscope within the colon may not know the rotational orientation of the position of the endoscope.

The endoscope may not know the position of an identified lesion from the laparoscopic view. A surgeon may be wiggle the tip of the scope against the colon and watch for the wiggle on the laparoscopic side. In this case, the orientation may be achieved through guesswork.

Lights may be configured on the sides of an endoscope. The lights may allow the laparoscopic view to orient to location of lesion including clocking (e.g., roll) of endoscope. The lights may provide laparoscopic-to-endoscopic support of resection of endoscope instruments. The lights may give the laparoscopic view the roll and clocking information associated with the endoscope. The working channel of the endoscope may be positioned at the desired position (e.g., 6:00 position) of the working channel to the lesion in the endoscope view. The laparoscopic view may have information of the endoscopic clocking. Laparoscopic support may be used through tissue positioning or full thickness lesion resection. Multiple color systems of LEDs may be projected circumferentially within the colon.

Laparoscopic to endoscopic light-based positional automation may be used. Based on the laparoscope view of the rotational position of the endoscope (e.g., as determine based on the pattern of projected lights), a tracking algorithm may create a positional zone in space. The zone may be laparoscopic-driven (e.g., automatically laparoscopic driven) by the robot. If the surgeon hits a ‘go to’ button on a controller interface (e.g., viewed on a console), the laparoscopic instrument may be moved into a zone near the lesion.

A time of flight distance sensor may confirm the distance to the colon and move the laparoscopic instrument a preset amount. If the preset amount is not achievable, the surgeon may receive operative communication (e.g., light, sound, haptic, etc.) of the failure. Manual motion may be allowed. For example, if the laparoscopic instrument is within the zone, an indication may be sent to the surgeon. Teleoperation of the instrument(s) may be resumed. At any time during the motion, the surgeon may move the teleoperation controls and resume surgeon-controlled motion.

One or more triggering events may initiate a calibration or indicate the increasing probability of error without recalibration. A device may redetermine an adjusted location of an EM sensor on the device if the device detects one or more triggering events. The triggering events may be temporal (e.g., recalibrate after a threshold time since the previous calibration). For example, recalibration may be triggered if the device detects that a maximum time since redetermining the adjusted location has passed. The triggering events may be positional. For example, the triggering events may be based on a change in positional relationship between the first surgical instrument and the second surgical instrument. The triggering event may be based on a distance that a surgical instruments has moved, a change of orientation or angle of a surgical instrument, and/or a change in velocity (e.g., position/time) of a surgical instrument.

The triggering events may be based on error terms. For example, the device may recalibrate if the device detects that an error associated with the adjusted location of the EM sensor is above an error threshold. The triggering events may be based on movements of a surgical device (e.g., quantity of movements, granularity of motion, and/or the like). The triggering events may be based on instrument configuration changes (e.g., articulation angle). The triggering events may be based on a risk or criticality associated with a procedure/operation step. For example, the device may recalibrate if the device detects that a criticality of an operation step is above a criticality threshold. The triggering events may be based on local monitoring of anatomy. For example, the device may recalibrate if the device detects that an anatomical structure in proximity to the first surgical instrument satisfies an anatomy condition (e.g., size of local passageways, variation from pre-operative imaging or plan, proximity to critical structures, etc.).

One or more types of recalibrations may be used. For example, the recalibration may be situational recalibration. External anatomic landmarks and/or proximity to known secondary sensing systems may be used to trigger recalibration. The recalibration may be continuous (e.g., on-the-fly continuous recalibration). Controlled/fixtured recalibration may be used. For example, robot articulation may be constrained within the trocar.

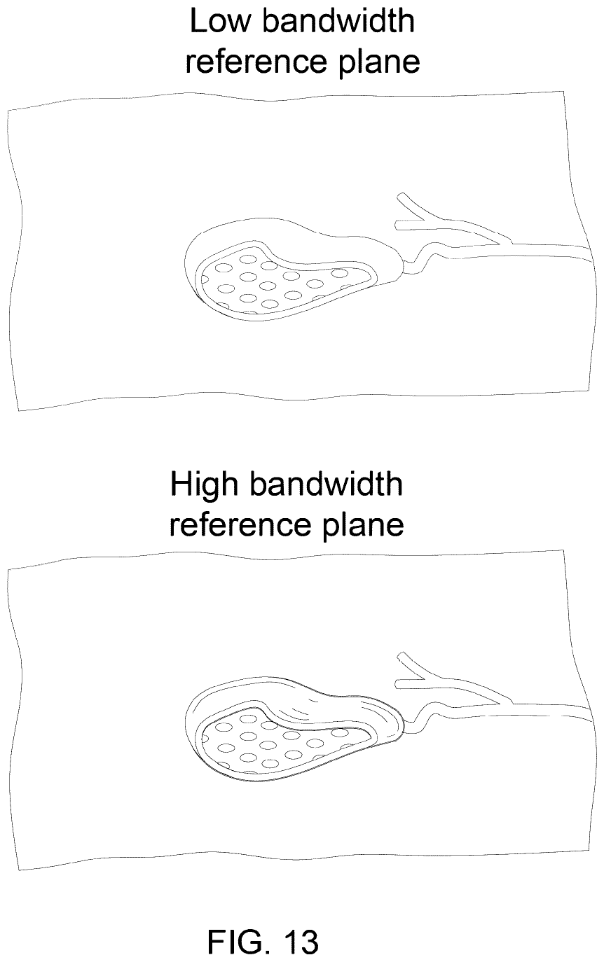

There may be a low bandwidth reference plane embedded within a high bandwidth reference plane, as illustrated in FIG. 13. If data becomes unavailable due to distortion, visual blockages such as hemostasis issues, camera fogging, device interference, reference plane may be reduced to the low bandwidth common reference plane (e.g., until coordinating system data becomes fully available). Low bandwidth imaging may use minimal data. Low bandwidth imaging may provide more general imaging until the high resolution data becomes available.

A vision system (e.g., the vision system that was not interfered with) may request specific data from another vision system (e.g., a vision system with an obscured view) in attempts to receive some information that has been labeled as critical or primary (e.g., low bandwidth information).

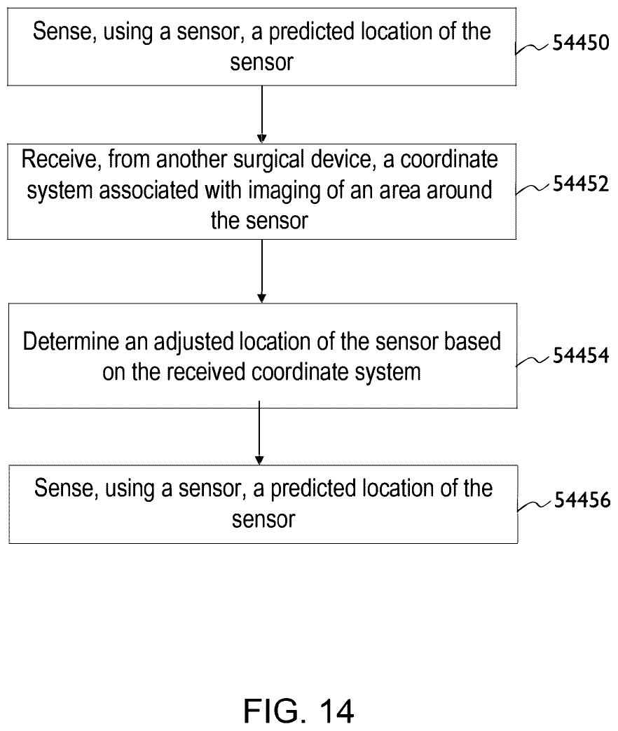

FIG. 14 illustrates an example method that may be performed by a surgical instrument. As shown at 54450, the method may involve sensing, using the EM sensor, a predicted location of the EM sensor. The predicted location may be distorted by an EM field. The method may involve, at 54452, receiving, from a second surgical instrument, a coordinate system associated with imaging of an area around the EM sensor. The method may involve, at 54454, determining an adjusted location of the EM sensor based on the received coordinate system. The method may involve, at 54456, outputting the adjusted location of the EM sensor.

What is claimed:

1. A first surgical instrument comprising a processor and an electromagnetic (EM) sensor, wherein the processor is configured to:
sense, using the EM sensor, a predicted location of the EM sensor, wherein the predicted location is distorted by an EM field;
receive, from a second surgical instrument, a coordinate system associated with imaging of an area around the EM sensor;
determine an adjusted location of the EM sensor based on the received coordinate system; and
output the adjusted location of the EM sensor.

2. The first surgical instrument of claim 1, wherein the processor is further configured to:
monitor distortion caused by proximity of the second surgical instrument;
identify a change in position of the second surgical instrument, wherein the distortion caused by proximity of the second surgical instrument is reduced due to the change in position; and
adjust the adjusted location of the EM sensor based on the reduced distortion.

3. The first surgical instrument of claim 1, wherein the processor is further configured to:
receive registration information of registered elements on a second surgical instrument;
monitor positional changes of the second surgical instrument based on the registration information; and
adjust the adjusted location of the EM sensor based on the monitored positional changes.

4. The first surgical instrument of claim 1, wherein:
the first surgical instrument is a laparoscopic probe;
the EM sensor is a magnetic tracker;
the second surgical instrument is a moveable CT machine;
the EM field causing distortion of the predicted location is created by proximity of the moveable CT machine to the first surgical instrument; and
the processor being configured to determine the adjusted location of the EM sensor based on the received coordinate system comprises the processor being configured to align expected anatomy associated with the predicted location with anatomy in the area around the EM sensor associated with the received coordinate system.

5. The first surgical instrument of claim 1, wherein:
the second surgical instrument is an ultrasound imaging probe,
the EM field causing distortion of the predicted location is created by proximity of the ultrasound imaging probe to a metal object; and
the processor being configured to determine the adjusted location of the EM sensor based on the received coordinate system comprises the processor being configured to align expected anatomy associated with the predicted location with anatomy in the area around the EM sensor associated with the received coordinate system.

6. The first surgical instrument of claim 1, wherein the processor is further configured to:
receive EM field monitoring information from a monitoring device located at a first distance from an EM device causing the predicted location to be distorted by the EM field, wherein the first distance is greater than a second distance between the first surgical instrument and the EM device; and wherein the adjusted location of the EM sensor is determined further based on the EM field monitoring information.

7. The first surgical instrument of claim 1, wherein the processor is further configured to redetermine the adjusted location of the EM sensor if the first surgical instrument detects one or more of:
a maximum time since redetermining the adjusted location has passed;
a change in positional relationship between the first surgical instrument and the second surgical instrument;
an error associated with the adjusted location of the EM sensor is above an error threshold;
a criticality of an operation step is above a criticality threshold; or
an anatomical structure in proximity to the first surgical instrument satisfies an anatomy condition.

8. The first surgical instrument of claim 1, wherein the adjusted location of the EM sensor is determined with respect to a global reference plane associated with an operating room in which the first surgical instrument is located.

9. A method performed by a first surgical instrument comprising an electromagnetic (EM) sensor, the method comprising:
sensing, using the EM sensor, a predicted location of the EM sensor, wherein the predicted location is distorted by an EM field;
receiving, from a second surgical instrument, a coordinate system associated with imaging of an area around the EM sensor;
determining an adjusted location of the EM sensor based on the received coordinate system; and
outputting the adjusted location of the EM sensor.

10. The method of claim 9, wherein the method further comprises:
monitoring distortion caused by proximity of the second surgical instrument;
identifying a change in position of the second surgical instrument, wherein the distortion caused by proximity of the second surgical instrument is reduced due to the change in position; and
adjusting the adjusted location of the EM sensor based on the reduced distortion.

11. The method of claim 9, wherein the method further comprises:
receiving registration information of registered elements on a second surgical instrument;
monitoring positional changes of the second surgical instrument based on the registration information; and
adjusting the adjusted location of the EM sensor based on the monitored positional changes.

12. The method of claim 9, wherein:
the first surgical instrument is a laparoscopic probe;
the EM sensor is a magnetic tracker;
the second surgical instrument is a moveable CT machine;
the EM field causing distortion of the predicted location is created by proximity of the moveable CT machine to the first surgical instrument; and
determining the adjusted location of the EM sensor based on the received coordinate system comprises aligning expected anatomy associated with the predicted location with anatomy in the area around the EM sensor associated with the received coordinate system.

13. The method of claim 9, wherein:
the second surgical instrument is an ultrasound imaging probe,
the EM field causing distortion of the predicted location is created by proximity of the ultrasound imaging probe to a metal object; and
determining the adjusted location of the EM sensor based on the received coordinate system comprises aligning expected anatomy associated with the predicted location with anatomy in the area around the EM sensor associated with the received coordinate system.

14. The method of claim 9, wherein the method further comprises:
receiving EM field monitoring information from a monitoring device located at a first distance from an EM device causing the predicted location to be distorted by the EM field, wherein the first distance is greater than a second distance between the first surgical instrument and the EM device; and wherein the adjusted location of the EM sensor is determined further based on the EM field monitoring information.

15. The method of claim 9, wherein the method further comprises redetermining the adjusted location of the EM sensor if the first surgical instrument detects one or more of:
a maximum time since redetermining the adjusted location has passed;
a change in positional relationship between the first surgical instrument and the second surgical instrument;
an error associated with the adjusted location of the EM sensor is above an error threshold;
a criticality of an operation step is above a criticality threshold; or
an anatomical structure in proximity to the first surgical instrument satisfies an anatomy condition.

16. The method of claim 9, wherein the adjusted location of the EM sensor is determined with respect to a global reference plane associated with an operating room in which the first surgical instrument is located.
